(19) United States
(12) Patent Application Publication
(10) Pub. No.: US 2020/0367824 A1
(43) Pub. Date: Nov. 26, 2020

(54) ADHESIVE LAYER STACK WITH OFFSET SEAMS

(71) Applicant: Verily Life Sciences LLC, South San Francisco, CA (US)

(72) Inventors: Sean Frick, San Francisco, CA (US); Roxana Heitz, Mountain View, CA (US)

(21) Appl. No.: 16/879,128

(22) Filed: May 20, 2020

Related U.S. Application Data
(60) Provisional application No. 62/850,975, filed on May 21, 2019.

(51) Int. Cl. A61B 5/00; A61B 50/30

(52) U.S. Cl. CPC A61B 5/6833; A61B 5/01; A61B 50/30

(57) ABSTRACT

A wearable device that includes a monitoring device; and an adhesive layer stack for adhering the monitoring device to the skin of a user. The adhesive layer stack includes a first adhesive layer comprising seam(s) forming a first pattern; and a second adhesive layer comprising seam(s) forming a second pattern; wherein the second adhesive layer is configured to be stacked vertically below the monitoring device and above the first adhesive layer; wherein the first adhesive layer is configured to be stacked vertically below the second adhesive layer and the monitoring device; and wherein: the first pattern is different from the second pattern; the first pattern is offset from the second pattern; and/or the first pattern has a first size and the second pattern has a second size that is different from the first size.

### CROSS-REFERENCE TO RELATED APPLICATION

This application claims the benefit of the filing date of, and priority to, U.S. Application No. 62/850,975, filed May 21, 2019, the entire disclosure of which is hereby incorporated herein by reference.

### TECHNICAL FIELD

The present disclosure relates generally to an adhesive layer stack with offset seams and methods of use with a body-mountable device.

### BACKGROUND

Body-mounted monitoring devices are often worn on the skin of a user. In some embodiments, the monitoring devices are worn by the user for multiple days via an adhesive layer that couples to the monitoring device and the skin of the user. The adhesive layer may break down or start to fail over time. Thus, the monitoring device is removed from the user, the adhesive layer is removed from the monitoring device, a new adhesive layer is applied to the monitoring device, and the monitoring device is then reapplied to the skin of the user. Removal of the adhesive layer is often problematic, as it may be difficult to fully remove the adhesive layer without damaging the components of the monitoring device such as batteries, sensors, circuitry, etc. Furthermore, the new adhesive layer requires time and care to apply, may be difficult to align, and has multiple parts that may be lost over time and use. Furthermore, since the device side and skin side of the adhesive often have different properties, there is also a risk to the user that the adhesive may be installed in the wrong direction.

### BRIEF DESCRIPTION OF THE DRAWINGS

Illustrative embodiments of the present disclosure will be described with reference to the accompanying drawings, of which:

FIG. 1 is a diagrammatic, perspective view of an example body-mountable device in accordance with at least one embodiment of the present disclosure, the body-mountable wearable device including a monitoring device coupled to an adhesive layer stack.

FIG. 2 is a diagrammatic, cross-sectional view of the body-mountable device of FIG. 1, in accordance with at least one embodiment of the present disclosure.

FIG. 3 is a diagrammatic, bottom view of the body-mountable device of FIG. 1, in accordance with at least one embodiment of the present disclosure.

FIG. 4 is a diagrammatic, bottom view of the body-mountable device of FIG. 3, in accordance with at least one embodiment of the present disclosure.

FIG. 5 is a diagrammatic, bottom view of the body-mountable device of FIG. 1 in which seams form a pattern in an adhesive layer of the adhesive layer stack, in accordance with another embodiment of the present disclosure.

FIG. 6 is a diagrammatic view of another pattern formed in an adhesive layer of the adhesive layer stack, in accordance with at least one embodiment of the present disclosure.

FIG. 7 is a diagrammatic, bottom view of the body-mountable device of FIG. 1, in accordance with another embodiment of the present disclosure.

FIG. 8 is a diagrammatic, bottom view of the body-mountable device of FIG. 7, in accordance with an embodiment of the present disclosure.

FIG. 9 is a diagrammatic, bottom view of the body-mountable device of FIG. 1, in accordance with another embodiment of the present disclosure.

FIG. 10 is a diagrammatic, bottom view of the body-mountable device of FIG. 9, in accordance with an embodiment of the present disclosure.

FIG. 11 is a perspective, exploded view of an example body-mountable device in accordance with at least one embodiment of the present disclosure.

FIG. 12 is a perspective, exploded view of a portion of the adhesive layer stack of FIG. 1, in accordance with an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

In accordance with at least one embodiment of the present disclosure, a body-mountable device is disclosed which can, for example, provide continuous monitoring of physical properties of a wearer of the body-mountable device. In an example, the device includes a monitoring device coupled to an adhesive layer stack. The adhesive layer stack includes multiple adhesive layers with offset seams so that removal of the lower-most adhesive layer automatically exposes another, new adhesive layer. Generally, the offset seams reduce stresses created during the removal of an old adhesive layer, so that these stresses do not cause the new layer to be also removed accidentally.

These descriptions are provided for example purposes only, and should not be considered to limit the scope of the body-mountable device. Certain features may be added, removed, or modified without departing from the spirit of the claimed subject matter.

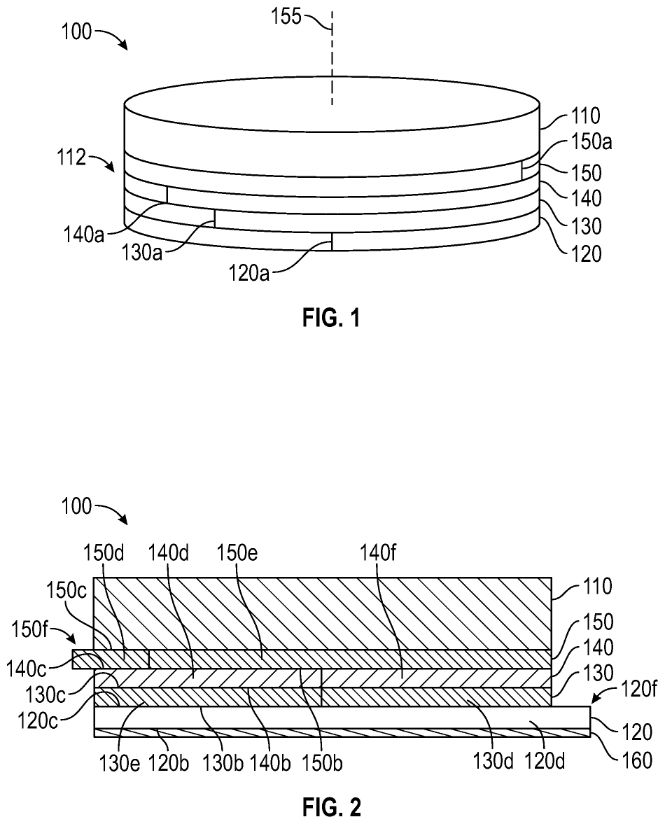

FIGS. 1-3 illustrate an example body-mountable device 100 in accordance with at least one embodiment of the present disclosure. In the example shown, the body-mountable device 100 includes a monitoring device 110 coupled to an adhesive layer stack 112. The adhesive layer stack 112 adheres the monitoring device 110 to the skin of the user. As illustrated, the adhesive layer stack 112 includes four removable adhesive layers 120, 130, 140, and 150, each with its own seam 120a, 130a, 140a, and 150a to aid the successive removal process of the adhesive layers from the stack 112. Generally, the adhesive layers 120, 130, 140, and 150 are stacked below or under the monitoring device 100 along a longitudinal axis 155 in FIG. 1. For ease of reference, this arrangement is also referred to as being stacked in the vertical direction with the adhesive layers 120, 130, 140, and 150 being stacked vertically under or below the monitoring device 100. Other numbers of adhesive layers could be included instead. In an example, the device includes between 1 and 10 adhesive layers. More than 10 adhesive layers is possible and considered here. Each adhesive layer forms a bottom surface 120b, 130b, 140b, and 150b (shown in FIG. 2) and an opposing top surface 120c, 130c, 140c, and 150c (shown in FIG. 2). The bottom-most surface of the adhesive stack 112, or 120b as illustrated in FIG. 2, may also be covered with a removable release liner 160 (only illustrated in FIG. 2). Optionally, the release liner 160 may be placed between each consecutively stacked layer, such as for example between surfaces 120c and 130b, between surfaces 130c and 140b, etc.

In an example, a patient may remove the removable release liner 160 from the bottom of the device 100, and then apply the device to his or her skin (e.g., for continuous glucose monitoring or other type of monitoring), with the bottom surface 120b of the lowest layer of the adhesive stack 112, the layer 120, in contact with his or her skin. The patient may wear the device 100 for a period of time, and then remove the device 100 (e.g., to bathe, sleep, swim, exercise, or after a predetermined period of time). When ready to mount the device 100 back on the user, the user may then remove the adhesive layer 120, and then re-apply the device 100 with the next layer, 130, in contact with his or her skin. This process may be repeated, with subsequent adhesive layers 130 and 140 being removed prior to each re-application of the device 100 to the patient's skin. This permits the monitor device 110 to be reused multiple times, without the user needing to apply a removable adhesive to the monitoring device 100, or perform maintenance and cleaning steps for a reusable adhesive.

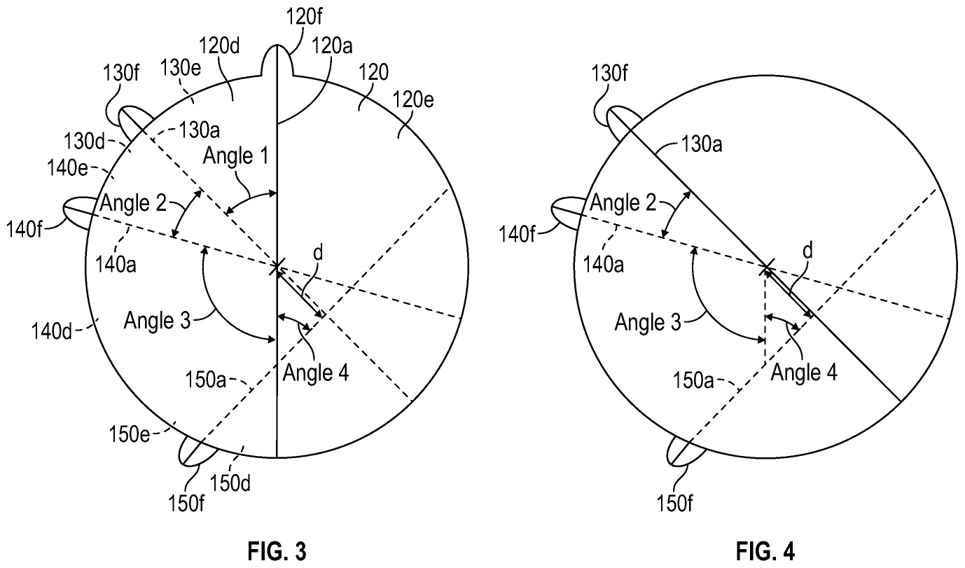

FIG. 3 is a diagrammatic, bottom view of the body-mountable device 100 when viewed along the longitudinal axis of the device 100. In this view, the bottom surface 120b of the bottom-most removable adhesive layer 120 is visible. In order to facilitate removal of adhesive layer 120, the layer 120 includes the seam 120a, such that the layer 120 is subdivided into two pieces: a left side 120d and a right side 120e, and also includes a tab 120f that projects outward from the device 100 and is also split by the seam 120a.

Also visible are tabs 130f, 140f, and 150f, which operate in the same manner as the tab 120f. Visible as hidden lines are seams 130a, 140a, and 150a, which split layers 130, 140, and 150, respectively, each into two sides 130d and 130e, 140d and 140e, and 150d and 150e. In an example, each seam 120a, 130a, 140a, and 150a has a different orientation, clock angle, or angular offset than each seam in the stack 112. However, in some embodiments, each seam 120a, 130a, 140a, and 150a has a different orientation, clock angle, or angular offset than the seam in the layers adjacent to it in the stack 112. This helps ensure that when one side of one layer is peeled up (e.g., side 120d), it does not inadvertently pull up a side of a different layer (e.g., side 130d) along with it. In the example shown in the FIG. 3, seam 120a is separated from seam 130a by Angle 1, while seam 130a is separated from seam 140a by Angle 2, and seam 140a is separated by seam 120a by Angle 3. As illustrated in FIG. 3, the seams 120a, 130a, and 140a intersect or cross at a center of the adhesive stack 112 but are angularly offset from each other by the angles 1, 2, and 3. In other words, butterfly patterns formed by the seams 120a, 130a, 140a are centered but angularly offset from each other. In at least one embodiment, some layers may have seams that are linearly offset from other seams in the stack 112 and/or from a center of the stack 112, so that stresses do not concentrate in the center of a layer while a side of a layer is being peeled off. In the example shown in FIG. 3, seam 150a is linearly offset from a center by distance d and angularly offset from the seam 120a by an Angle 4. That is, when a first seam extends through a center of the adhesive stack, then a second seam that has a linear offset from the first seam does not extend through the center of the adhesive stack, and the first and second seams cross or intersect at a location that is offset from the center of the adhesive stack 112. In other examples, different layers may be offset by different amounts, or by the same amount at different clock angles. For purposes of this document, this arrangement of seams will be known as a “butterfly” pattern.

In an example, a user who wanted to remove the removable adhesive layer 120 would grip the right side of the tab 120f and pull upward, or along the longitudinal axis in a direction away from the monitoring device 110, such that the right side 120e of the removable adhesive layer 120 is peeled away from the device 100. The user would then grip the left side of tab 120f and pull upward such that the left side 120d of the removable adhesive layer 120 is peeled from the device 100. Removing the adhesive layer 120 exposes the adhesive layer 130, as illustrated in FIG. 4. This “butterfly seam” arrangement (i.e., the subdivision of an adhesive layer into two pieces using a seam) makes it possible to remove only half of the adhesive layer 120 at a time, thus requiring only half of the total pulling force. This helps prevent the other layers 130, 140, and 150 from separating inadvertently when layer 120 is removed.

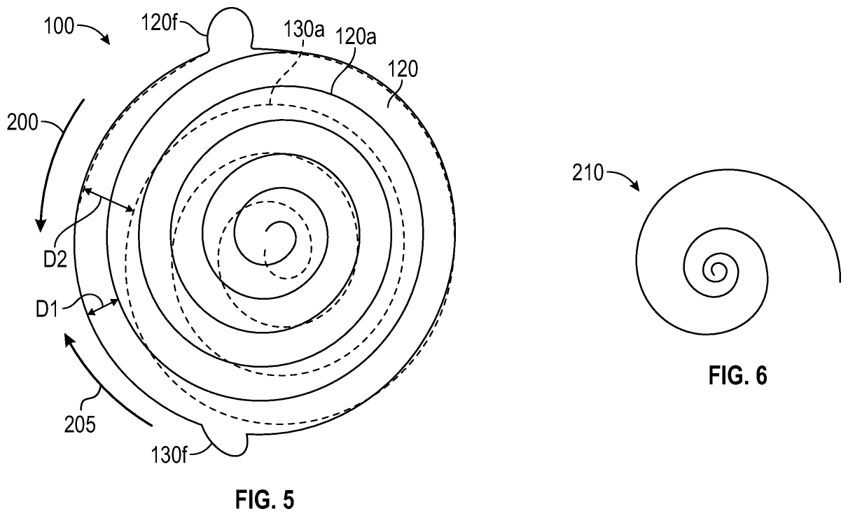

FIG. 5 is a diagrammatic, bottom view of an example multi-use disposable electronic skin patch in accordance with at least one embodiment of the present disclosure. Instead of a butterfly pattern, the seam 120a forms a spiral arm with a spiral arm dimension D1. As illustrated in FIG. 5, the spiral arm dimension is generally consistent to form a spiral pattern. As such, the seam 120a is a spiral seam that permits the first adhesive layer 120 to be peeled off gradually, without a large application of force across a large area, when the tab 120f is pulled. Also visible is tab 130f and the seam 130a as a hidden line. Adhesive layers 140 and 150 are not shown in FIG. 5. The seam 130a forms a spiral arm with a spiral arm dimension D2. As illustrated, the spiral arm dimension D2 is generally consistent but different from the spiral arm dimension D2 to form another spiral pattern that is of a different size than the spiral pattern associated with adhesive layer 120. Moreover, the spiral direction of adhesive layer 120 is in the direction 200 while the direction of the adhesive layer 130 is in the direction 205, which is opposite to the direction 200. While the seams 120a and 130a intersect or cross, this prevents the seams 120a and 130a from lining up, which thereby prevents the removal of layer 120 from accidentally removing part or all of layer 130 when a pulling force is applies to tab 120f.

FIG. 6 is an illustration of another spiral pattern 210 formed by the seam 120a and/or the seam 130a, according to another example embodiment. As illustrated, the spiral pattern 210 has a variable spiral arm dimension. In an example, by changing the size of spiral arm dimension, direction of the spiral pattern, and/or between consistent spiral dimension and/or variable spiral arm dimension, from one layer to the next, the chance of accidental removal of other layers is minimized or reduced.

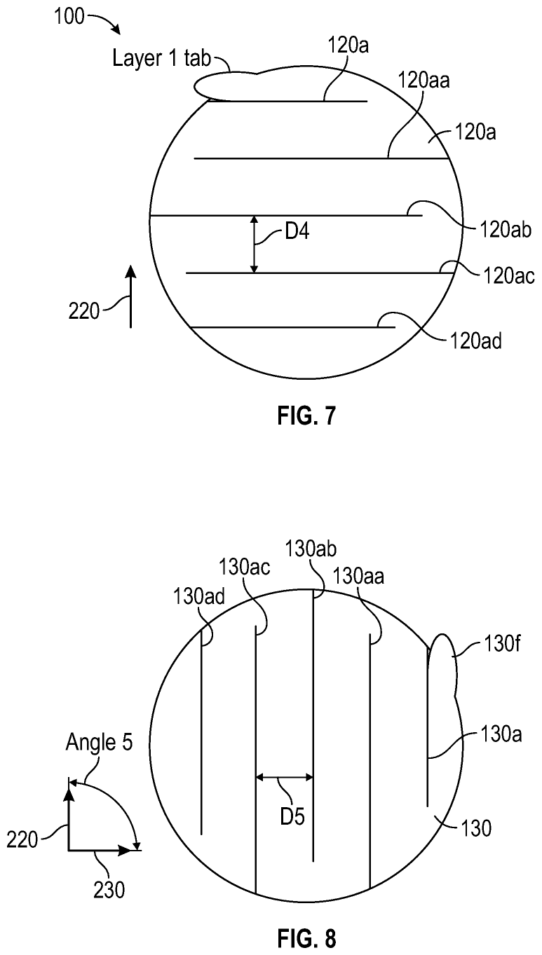

FIG. 7 is a diagrammatic, bottom view of an example adhesive layer 120 in accordance with at least one embodiment of the present disclosure. In this example, seam 120a in layer 120 is one of many seams 120aa, 120ab, 120ac, and 120ad that forms a double-back pattern. Generally, the seams 120a, 120aa, 120ab, 120ac, and 120ad are horizontal and spaced along the adhesive layer 120 in a direction 220 by a distance D4. The seams 120a, 120ab, and 120ad intersect with a first edge of the adhesive layer 120 but are spaced from an opposite second edge of the adhesive layer while the seams 120aa and 120ac intersect with second edge but are spaced from the first edge. This forms a continuous double-back strip of adhesive layer. Pulling on the tab 120f in an upward direction results in portions, defined by the seams 120a, 120aa, 120ab, 120ac, and 120ad, to be removed from the adhesive stack 112. Generally, the double-back pattern formed in the adhesive layer 120 is oriented in the direction 220. FIG. 8 is a diagrammatic, bottom view of an example adhesive layer 130. The adhesive layer 130 is almost identical to the adhesive layer 120 of FIG. 7 such that seam 130a in layer 130 is one of many seams 130aa, 130ab, 130ac, and 130ad that form a double-back pattern. However, the seams 130a, 130aa, 130ab, 130ac, and 130ad are generally horizontal and spaced along the adhesive layer 130 in a direction 230 by a distance D5. The seams 130a, 130ab, and 130ad intersect with a first edge of the adhesive layer 130 but are spaced from an opposite second edge of the adhesive layer while the seams 130aa and 130ac intersect with second edge but are spaced from the first edge. This forms a continuous double-back strip of adhesive layer. Pulling on the tab 130f in an upward direction results in portions, defined by the seams 130a, 130aa, 130ab, 130ac, and 130ad, to be removed from the adhesive stack 112. The directions 220 and 230 are angularly offset or rotated by an Angle 5, as illustrated in FIG. 8. As illustrated, the distance D4 is the same as the distance D5, but in some embodiments the distance D4 is different from the distance D5. The directions 220 and 230 are offset by a 90 degree angle as illustrated, but could be offset by any number of angles such as 30 degrees, 45 degrees, 120 degrees, 220 degrees, etc. In some embodiments, the directions 220 and 230 (and patterns associated with 120 and 130) are angularly offset by an angle and the distances D4 and D5 are different.

Generally, the patterns formed in or associated with the adhesive layers 120, 130, 140, and 150 vary from one another using any combination and size of a spiral pattern, butterfly pattern, and cross-back pattern.

To prevent layers from peeling apart inadvertently, some embodiments also include a progressively stronger adhesive on the layers as the layers are stacked closer to the monitoring device 110, such that the adhesive between surfaces 130c and 140b has a greater peel strength than the adhesive between surfaces 120c and 130b. Such adhesive strength increases may be achieved for example by varying the thickness of the adhesive or the ratio of its components according to methods that are known in the art. Alternatively, these differences in peel strength can be achieved by varying the effectiveness of a release coating disposed on the surfaces 120c, 130c, and 140c. Such differences in release coating effectiveness can be achieved by varying the thickness, composition, or percent coverage of the release coating.

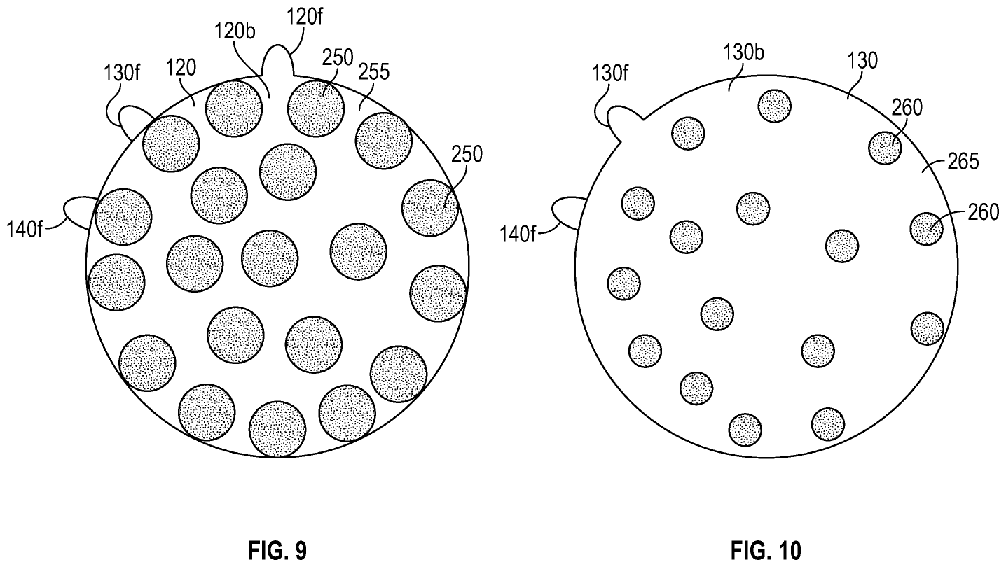

In one embodiment, the differences in peel strength or adhesive strength between layers 120, 130, 140, and 150 is based on the differences of an adhesive strength and/or a surface area covered or including an adhesive. For example, FIG. 9 is a diagrammatic, bottom view of an example device 100 including the layer 120 in accordance with at least one embodiment of the present disclosure. In this configuration, the layer 120 includes areas of adhesive 250 (e.g., “adhesive islands”) interspersed with non-adhesive areas 255 across the surface 120b of adhesive layer 120. FIG. 10 is a diagrammatic, bottom view of an example device 100 with the layer 120 removed to show the layer 130 in accordance with at least one embodiment of the present disclosure. In this configuration, the layer 130 includes areas of adhesive 260 (e.g., “adhesive islands”) interspersed with non-adhesive areas 265 across the surface 130b of adhesive layer 130. As illustrated, layer 120 provides larger adhesive islands 250 than the adhesive islands 260 of layer 130. In some embodiments, the adhesive islands 250 and the adhesive islands 260 have different adhesive strength. For example, the adhesive islands 250 include a stronger adhesive than the adhesive in the adhesive islands 260. Not only can the adhesive islands 250 and 260 have different sizes and different strengths, the number of adhesive islands 250 may be different than the number of adhesive islands 260. The number, size, and strength can change in each of the layers 120, 130, 140, and 150 such that the adhesive strength increases or decreases as the adhesive layer is closer to the monitoring device 110. In the FIGS. 9 and 10, the adhesive islands 250 and 260 are shown as circular discs, although other shapes could be employed. In the adhesive islands configuration, each adhesive layer has different sized adhesive islands, a different number of adhesive islands, and/or adhesive islands made of different adhesive strength than each subsequent layer toward the topmost layer (e.g., adhesive layer 150). It is noted that smaller islands may also include a stronger adhesive, and that larger islands may also include a weaker adhesive, while serving the goal of making each subsequent layer require a larger removal force than the ones before it, thus reducing the risk of accidentally removing more than one adhesive layer when disposing of a used adhesive layer. At any given time, this allows the bottom-most layer to be removed more easily, by ensuring that an amount of force able to remove the bottom-most layer will not also remove any of the higher layers, or else will do so before any removal or weakening of higher layers can occur.

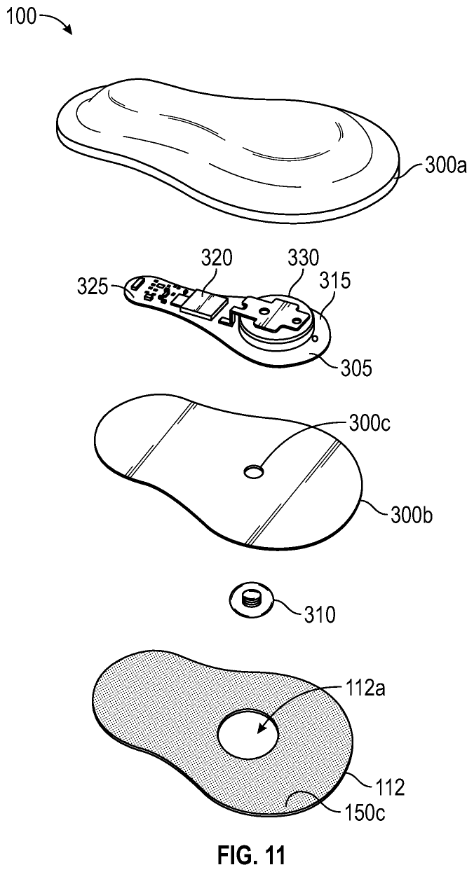

FIG. 11 is an exploded perspective of one embodiment of the device 100. In some embodiments, the monitoring device 110 includes an upper housing 300a and a complimentary lower housing 300b that is configured to couple to the upper housing 300a. In some embodiments, the device 100 is a patch-like device that is attached, via the adhesive stack 112, to the torso of a patient. When the housings 300a and 300b are coupled together, a chamber is formed that houses a control system 305. In an example embodiment, the housings 300a and 300b are elastomeric housings. A thermal contact 310 is coupled to the lower housing 300b and is in communication with the control system 305 via an opening 300c formed in the lower housing 300b. In some embodiments, the thermal contact 310 is replaced with another type of contact that is capable of monitoring any number of properties of the user based on the proximity of the contact to the skin of the user. The adhesive layer stack 112 is in contact with the lower housing 300b.

In some embodiments, the control system 305 includes a substrate 315, one or more sensors 320, a microcontroller 325, and a power source 330. In some implementations, the substrate 315 can be a circuit board or printed circuit board (PCB). Additional or fewer components are possible. For example, the control system 305 may include other biometric sensors that are in communication with the microcontroller 325. Generally, when the device 100 includes a thermal contact, the one or more sensors 320 include a temperature sensor and it is in communication with the microcontroller 325 and the thermal contact 310. In some embodiments, an opening 112a is formed in the adhesive stack 112 and through the adhesive layers 120, 130, 140, and 150 such that the thermal contact 310 extends through the openings 112a to contact the skin of the user and/or be positioned next to the skin of the user without an adhesive layer extending between the thermal contact 310 and the skin of the patient. In some embodiments, the opening 112a has a diameter that is greater than the outer diameter of the thermal contact 310. However, in other embodiments, the diameter of the opening 112a is equal to or less than the outer diameter of the thermal contact 310. In some examples, the thermal contact 310 facilitates physical measurement of properties of the body of the wearer (e.g., of the skin at the external body surface). As shown, the thermal contact 310 is a circular, rounded contact configured to protrude from the lower housing 300b and to make electrical and/or thermal contact with the skin of the external body surface of the wearer. In some embodiments, the thermal contact 310 extends between about 0.05 cm and about 0.2 cm from the lower housing 300b. In some embodiments, the contact 310 is in electrical contact with the one or more sensors 320 such that a galvanic skin response (“GSR”) of the skin at the external body surface can be detected by the device 110. Generally, the thermal contact 310 or other contact and the one or more sensors 320 are positioned and configured to continuously monitor and detect a temperature of a patient or other property of the patient or user. In other embodiments, the thermal contact 310 and the one or more sensors 320 are positioned and configured to intermittently monitor the temperature of the patient. In some embodiments, the thermal contact 310 is a metal disc.

As illustrated in FIG. 11, the adhesive stack 112 can have a footprint that is a shape other than a circular shape. As illustrated, the adhesive stack 112 has a footprint that is shaped similarly to a pear.

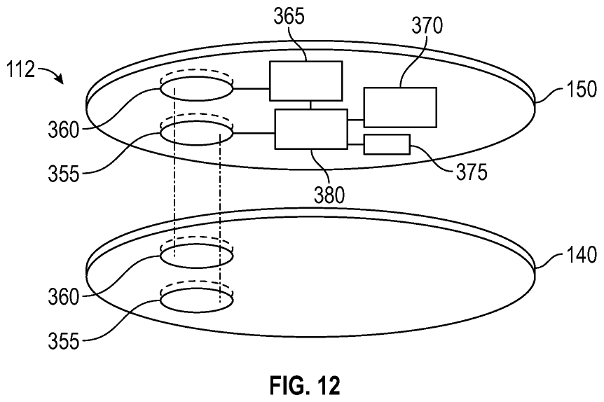

In other embodiments and as illustrated in FIG. 12, the opening 112a in the adhesive stack 112 is omitted and each adhesive layer 140 and 150 (seams not shown) includes contacts 355 and 360 that are capable of making contact with the contacts 355 and 360 in the adhesive layers stacked above and below it. In some embodiments, layer 150 includes a processor 365, a memory 370, a communication device 375, and a thermal sensor 380 in communication with each other and in communication with the contacts 355 and 360 within each of the layers 140 and 150. In some embodiments, the layers 120 and 130 are identical to the layer 140 as illustrated in FIG. 12. In some embodiments, the contacts 355 and 360 create a sensor interface or thermal contact to couple the thermal sensor 380 to the skin of the user. Removal of the layer 130 (not shown) exposes the contacts 355 and 360 of the layer 140 and so on. As such, the contacts 355 and 360, individually or in combination, are the thermal contact 310. In some embodiments, the processor 365, memory 370, the communication device 375, and the thermal sensor 380 are omitted and the contacts 355 and 360 are coupled to, or function as, the thermal contact 310, which is operably coupled to the control system 305.

In other embodiments, the thermal contact 310, or the contacts 355 and 360, may instead be some other type of measurement device, such as a continuous glucose monitor (CGM). In the case of a CGM, the CGM monitors glucose levels using measurements taken just beneath the skin using any well-known method. In this configuration, removable portions of the sensor interface formed via the contacts 355 and 360 connect the CGM to the skin of the patient.

In some embodiments, each layer 120, 130, 140, and 150 includes electrical contact posts that are capable of making contact with electrical contact posts in the adhesive layers stacked above and below it. In electrical association with the posts and are insulated conductive traces leading to a sensor. In some embodiments, two posts connect to two traces, which connect to a single sensor. However, a person of ordinary skill in the art will understand that any number of posts traces, sensors, and components (as described above) may be used in each adhesive layer, or that each adhesive layer may be a self-sufficient device containing its own power sources and data transmitters or transceivers, or that each adhesive layer may be a passive device that includes no electrical or electronic devices. In some embodiments, each adhesive layer includes two electrical contact receivers that may be electrically connected with similarly positioned electrical contacts on the bottom surface of the adhesive layer above it. In this manner, a plurality of adhesive layers may be stacked one on top of another against the monitor device 110 while maintaining electrical or data communication with the monitor device 110. In some embodiments, the sensors of the multi-use disposable electronic skin patch (e.g., gas sensors, accelerometers, radiation sensors) exist entirely within the monitor device 110 and may not require direct contact with human skin, in which case the adhesive layers 120, 130, 140, and 150 serve only as passive carriers for the adhesive that holds the monitor device 110 onto the patient's body, and as such do not perform any sensing, processing, or communication operations of their own.

In some embodiments, each of the surfaces 120b, 130b, 140b, and 150b includes a patient contact adhesive (i.e., skin contact adhesive), and such components as are necessary to permit the monitoring device 110 to read patient data through the adhesive layer. Such components may include but are not limited to hollows, voids, through-holes, windows, electrical contacts, electrical traces, batteries, electronic components, processors, antennas, and sensors.

Possible advantages of this solution include ease of use, defined number of uses, and a device 100 that can be removed from the skin but still reused, without the need to remove and reapply an adhesive, or the drawbacks associated with a reusable adhesive.

In some embodiments, the top surfaces 120c, 130c, and 140c do not include an adhesive. Instead the top surfaces 120c, 130c, 140c may incorporate release coatings that perform the same function of making it possible to remove the layers 120, 130, and 140 from the stack 112 without damaging the adhesive of the layer above it, and without damaging any layer or component of the device other than perhaps the adhesive layer being removed.

In some embodiments, the uppermost adhesive layer 150 is removable from the monitoring device 110 and a new adhesive stack can be attached to the monitoring device 110.

Based on design considerations, different adhesive layers within a single device may have different configurations as described above, or alternative configurations that achieve the described result.

Depending on the implementation, a disposable release liner 160 may also be positioned between each of the layers 120, 130, 140, and 150. Generally, the disposable release liner is not intended to be against the skin. Rather, it has a release coating on its top surface and an adhesive coating on its bottom surface that is intended to bind it to the adhesive layer beneath it.

In some embodiments, the adhesive stack 112 provides a plurality of adhesive layers configured to release at progressively higher forces, with geometry appropriate to aid in the release as described hereinabove, or a release sequence wherein each time the device is peeled off of the user's skin, the old layer of adhesive is left behind on the skin and removed separately from the device 100. In each of these examples, the end result is that the user can remove the device (e.g., to bathe, sleep, swim, or exercise) and also remove a layer of adhesive from the device, such that the device may be reapplied and reused with the next layer of adhesive.

A number of variations are possible on the examples and embodiments described above. Accordingly, the logical operations making up the embodiments of the technology described herein are referred to variously as operations, steps, objects, elements, components, layers, modules, or otherwise. Furthermore, it should be understood that these may occur in any order, unless explicitly claimed otherwise or a specific order is inherently necessitated by the claim language.

In some implementations, the multiple disposable adhesive layers may include sensors or other electronic devices or non-electronic devices that interact with the monitor layer or other layers, or that are independent. It should further be understood that the described technology may be employed in veterinary medicine as well as human medicine, and that multiple multi-use removable patches of the same or different types could be worn by a patient at the same time.

Data communication, if any, between the monitor device 110 and adhesive layers 120-150 may be through numerous methods or protocols. For example, a resistive analog sensor may be both powered and interrogated by placing a voltage across it using two wires. Serial communication protocols may include but are not limited to SPI, I²C, RS-232, RS-485, CAN, Ethernet, ARINC 429, MODBUS, MIL-STD-1553, or any other suitable method or protocol. Depending on the implementation, parallel protocols may also be used, including but not limited to ISA, ATA, SCSI, PCI, IEEE-488, IEEE-1284, and other suitable protocols. Where appropriate, serial and parallel communications may be bridged by a UART, USART, or other appropriate subsystem. Depending on the implementation, the monitor device 110 and adhesive layers 120, 130, 140, and 150 may also communicate using cable interfaces such as USB, micro USB, Lightning, or FireWire, or wirelessly via Bluetooth, Wi-Fi, ZigBee, Li-Fi, or cellular data connections such as 2G/GSM, 3G/UMTS, 4G/LTE/WiMax, or 5G. In some embodiments, the monitor device 110 may be configured to communicate with a remote server, or with a nearby device such as a laptop, tablet, or handheld device, or may include a display capable of showing status variables and other information.

It is noted that the bottom surface of the monitor device 110 may also serve as a patient contact surface, including whatever adhesives and sensors are required to serve that function. In this regard, the monitoring device 110 can be applied directly to the skin of the user without one of the adhesive layers 120, 130, 140, and 150 positioned between the monitor device 110 and the skin of the user. Alternatively, in some embodiments a non-removable adhesive layer (aka, a carrier layer), which for convenience may be similar or identical to the removable adhesive layers, may be permanently attached to the monitor device 110 (e.g., using a permanent adhesive).

In an example, the device 100 may be used to track a patient's levels of glucose, insulin, hormones, nutrients, drugs, alcohol, drug or alcohol metabolites, skin galvanic response, heart rate, blood oxygen, and other health-related or non-health-related variables.

In an example, the adhesive layers 120, 130, 140, and 150 are made of a flexible material, which may also be a breathable material that allows moisture evaporation. In some embodiments, the monitor device 110 is a rigid device. In other embodiments, the monitor device 110 comprises a flex circuit or other flexible device.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the multi-use disposable electronic skin patch. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

Generally, any creation, storage, processing, and/or exchange of user data associated the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In other embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

A wearable device has been disclosed that includes: a monitoring device; and an adhesive layer stack for adhering the monitoring device to the skin of a user, the adhesive layer stack including: a first adhesive layer including seam(s) forming a first pattern; and a second adhesive layer including seam(s) forming a second pattern; wherein the second adhesive layer is configured to be stacked vertically below the monitoring device and above the first adhesive layer; wherein the first adhesive layer is configured to be stacked vertically below the second adhesive layer and the monitoring device; and wherein: the first pattern is different from the second pattern; the first pattern is offset from the second pattern; and/or the first pattern has a first size and the second pattern has a second size that is different from the first size. In one embodiment, the first pattern is different from the second pattern. In one embodiment, the first pattern is one of a spiral pattern, a double-back pattern, and a butterfly pattern; and wherein the second pattern is another of the spiral pattern, the double-back pattern, and the butterfly pattern. In one embodiment, the first pattern is offset from the second pattern. In one embodiment, the first pattern is linearly offset from the second pattern and/or angularly offset from the second pattern. In one embodiment, the first pattern is centered with the second pattern and rotated relative to the second pattern such that the first pattern is angularly offset from the second pattern. In one embodiment, the first pattern has the first size and the second pattern has the second size that is different from the first size. In one embodiment, the first adhesive layer is removable from the adhesive layer stack to expose the second adhesive layer. A monitoring device adapted to adhere to the skin of the user via the adhesive layer stack, the monitoring device including: a microcontroller; a power source in communication with the microcontroller; and one or more sensors in communication with the microcontroller; wherein the adhesive layer stack is coupled to the monitoring device; and wherein the first adhesive layer is configured to be in contact with the skin to adhere the monitoring device to the skin of the user such that the one or more sensors monitors a measurement via the skin of the user.

An adhesive layer stack with offset seams for adhering a monitoring device to the skin of a user has been disclosed with the adhesive layer stack including: a first adhesive layer including a first seam; and a second adhesive layer including a second seam; wherein the second adhesive layer is configured to be stacked vertically below the monitoring device and above the first adhesive layer; wherein the first adhesive layer is configured to be stacked vertically below the second adhesive layer and the monitoring device; and wherein, when viewed in the vertical direction, the first seam is offset from the second seam. In one embodiment, when viewed in the vertical direction, the first seam is angularly offset from the second seam. In one embodiment, when viewed in the vertical direction, the first adhesive layer is linearly offset from the second seam. In one embodiment, when viewed in the vertical direction, the first seam forms a first spiral arm; wherein, when viewed in the vertical direction, the second seam forms a second spiral arm; and wherein: the first spiral arm has a generally consistent first spiral arm dimension and the second spiral arm has a generally consistent second spiral arm dimension that is different from the first spiral arm dimension; the first spiral arm has a variable spiral arm dimension and the second spiral arm has a variable spiral arm dimension; and/or the first spiral arm spirals in a first direction and the second spiral arm spirals in a second direction that is opposite the first direction. In one embodiment, when viewed in the vertical direction, the first seam is one of a first plurality of seams formed in the first adhesive layer that forms a first double-back pattern within the first adhesive layer; wherein, when viewed in the vertical direction, the second seam is one of a second plurality of seams formed in the second adhesive layer that forms a second double-back pattern within the second adhesive layer; and wherein the first double-back pattern is angularly offset from the second double-back pattern. In one embodiment, the first adhesive layer is removable from the adhesive layer stack to expose the second adhesive layer. A monitoring device adapted to adhere to the skin of the user via the adhesive layer stack, the monitoring device including: a microcontroller; a power source in communication with the microcontroller; and one or more sensors in communication with the microcontroller; wherein the adhesive layer stack is coupled to the monitoring device; and wherein the first adhesive layer is configured to be in contact with the skin to adhere the monitoring device to the skin of the user such that the one or more sensors monitors a measurement via the skin of the user.

A method of removing a first adhesive layer from a stack of adhesive layers that is attached to monitoring device is disclosed, the method including: pulling on a tab portion of the first adhesive layer; in response to pulling on the tab portion, separating a first portion of the first adhesive layer from the stack of adhesive layers; wherein the first portion is defined by at least one seam formed in the first adhesive layer; and wherein the at least one seam formed in the first adhesive layer forms a first pattern in the first adhesive layer; after separating the first portion of the first adhesive layer from the stack of adhesive layers, exposing a second adhesive layer that forms the stack of adhesive layers; wherein the second adhesive layer includes at least one seam to form a second pattern in the second adhesive layer; and wherein: the first pattern is different from the second pattern; the first pattern is offset from the second pattern; and/or the first pattern has a first size and the second pattern has a second size that is different from the first size. In one embodiment, exposing the second adhesive layer is in response to separating the first portion of the first adhesive layer from the stack of adhesive layers. In one embodiment, the first pattern is one of a spiral pattern, a double-back pattern, and a butterfly pattern. In one embodiment, the first pattern is offset from the second pattern. In one embodiment, the first pattern has a first size and the second pattern has a second size that is different from the first size.

The above specification, examples and data provide a complete description of the structure and use of example embodiments of the body-mountable device as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. For example, the disclosed technology could be applied for compliance monitoring in substance abuse treatment programs. Additionally, the disclosed technology could be applied to body-mountable devices that do not monitor substance levels in the skin, but rather substance levels in the air, light or UV levels, or other useful information. Alternatively, or in addition, the body-mountable device could perform useful functions such as motion tracking, communication, or display of health data or other information. Alternatively, the disclosed technology could be applied to non-electronic devices such as passive drug delivery patches, recreational stickers, or other passive devices, without departing from the spirit or subject matter of the present disclosure.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

In several example embodiments, the elements and teachings of the various illustrative example embodiments may be combined in whole or in part in some or all of the illustrative example embodiments. In addition, one or more of the elements and teachings of the various illustrative example embodiments may be omitted, at least in part, and/or combined, at least in part, with one or more of the other elements and teachings of the various illustrative embodiments.

Any spatial references such as, for example, “upper,” “lower,” “above,” “below,” “between,” “bottom,” “vertical,” “horizontal,” “angular,” “upwards,” “downwards,” “side -to-side,” “left-to-right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In several example embodiments, while different steps, processes, and procedures are described as appearing as distinct acts, one or more of the steps, one or more of the processes, and/or one or more of the procedures may also be performed in different orders, simultaneously, and/or sequentially. In several example embodiments, the steps, processes and/or procedures may be merged into one or more steps, processes, and/or procedures.

In several example embodiments, one or more of the operational steps in each embodiment may be omitted. Moreover, in some instances, some features of the present disclosure may be employed without a corresponding use of the other features. Moreover, one or more of the above-described embodiments and/or variations may be combined in whole or in part with any one or more of the other above-described embodiments and/or variations.

The phrase “at least one of A and B” should be understood to mean “A, B, or both A and B.” The phrase “one or more of the following: A, B, and C” should be understood to mean “A, B, C, A and B, B and C, A and C, or all three of A, B, and C.” The phrase “one or more of A, B, and C” should be understood to mean “A, B, C, A and B, B and C, A and C, or all three of A, B, and C.”

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims. In the claims, any means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What is claimed is:

1. A wearable device comprising:
a monitoring device; and
an adhesive layer stack for adhering the monitoring device to the skin of a user, the adhesive layer stack comprising:
a first adhesive layer comprising seam(s) forming a first pattern; and
a second adhesive layer comprising seam(s) forming a second pattern;
wherein the second adhesive layer is configured to be stacked vertically below the monitoring device and above the first adhesive layer;
wherein the first adhesive layer is configured to be stacked vertically below the second adhesive layer and the monitoring device; and
wherein:
the first pattern is different from the second pattern;
the first pattern is offset from the second pattern; and/or
the first pattern has a first size and the second pattern has a second size that is different from the first size.

2. The wearable device of claim 1, wherein the first pattern is different from the second pattern.

3. The wearable device of claim 1,
wherein the first pattern is one of a spiral pattern, a double-back pattern, and a butterfly pattern; and
wherein the second pattern is another of the spiral pattern, the double-back pattern, and the butterfly pattern.

4. The wearable device of claim 1, wherein the first pattern is offset from the second pattern.

5. The wearable device of claim 4, wherein the first pattern is linearly offset from the second pattern and/or angularly offset from the second pattern.

6. The wearable device of claim 1, wherein the first pattern is centered with the second pattern and rotated relative to the second pattern such that the first pattern is angularly offset from the second pattern.

7. The wearable device of claim 1, wherein the first pattern has the first size and the second pattern has the second size that is different from the first size.

8. The wearable device of claim 1, wherein the first adhesive layer is removable from the adhesive layer stack to expose the second adhesive layer.

9. A monitoring device adapted to adhere to the skin of the user via the adhesive layer stack of claim 1, the monitoring device comprising:
a microcontroller;
a power source in communication with the microcontroller; and
one or more sensors in communication with the microcontroller;
wherein the adhesive layer stack is coupled to the monitoring device; and
wherein the first adhesive layer is configured to be in contact with the skin to adhere the monitoring device to the skin of the user such that the one or more sensors monitors a measurement via the skin of the user.

10. An adhesive layer stack with offset seams for adhering a monitoring device to the skin of a user, the adhesive layer stack comprising:
a first adhesive layer comprising a first seam; and
a second adhesive layer comprising a second seam;
wherein the second adhesive layer is configured to be stacked vertically below the monitoring device and above the first adhesive layer;
wherein the first adhesive layer is configured to be stacked vertically below the second adhesive layer and the monitoring device; and
wherein, when viewed in the vertical direction, the first seam is offset from the second seam.

11. The adhesive layer stack of claim 10,
wherein, when viewed in the vertical direction, the first seam is angularly offset from the second seam.

12. The adhesive layer stack of claim 10,
wherein, when viewed in the vertical direction, the first adhesive layer is linearly offset from the second seam.

13. The adhesive layer stack of claim 10,
wherein, when viewed in the vertical direction, the first seam forms a first spiral arm;
wherein, when viewed in the vertical direction, the second seam forms a second spiral arm; and
wherein:
the first spiral arm has a generally consistent first spiral arm dimension and the second spiral arm has a generally consistent second spiral arm dimension that is different from the first spiral arm dimension;
the first spiral arm has a variable spiral arm dimension and the second spiral arm has a variable spiral arm dimension; and/or
the first spiral arm spirals in a first direction and the second spiral arm spirals in a second direction that is opposite the first direction.

14. The adhesive layer stack of claim 10,
wherein, when viewed in the vertical direction, the first seam is one of a first plurality of seams formed in the first adhesive layer that forms a first double-back pattern within the first adhesive layer;
wherein, when viewed in the vertical direction, the second seam is one of a second plurality of seams formed in the second adhesive layer that forms a second double-back pattern within the second adhesive layer; and
wherein the first double-back pattern is angularly offset from the second double-back pattern.

15. The adhesive layer stack of claim 10, wherein the first adhesive layer is removable from the adhesive layer stack to expose the second adhesive layer.

16. A monitoring device adapted to adhere to the skin of the user via the adhesive layer stack of claim 10, the monitoring device comprising:
a microcontroller;
a power source in communication with the microcontroller; and
one or more sensors in communication with the microcontroller;
wherein the adhesive layer stack is coupled to the monitoring device; and
wherein the first adhesive layer is configured to be in contact with the skin to adhere the monitoring device to the skin of the user such that the one or more sensors monitors a measurement via the skin of the user.

17. A method of removing a first adhesive layer from a stack of adhesive layers that is attached to monitoring device, the method comprising:
pulling on a tab portion of the first adhesive layer;
in response to pulling on the tab portion, separating a first portion of the first adhesive layer from the stack of adhesive layers;
wherein the first portion is defined by at least one seam formed in the first adhesive layer; and
wherein the at least one seam formed in the first adhesive layer forms a first pattern in the first adhesive layer;
after separating the first portion of the first adhesive layer from the stack of adhesive layers, exposing a second adhesive layer that forms the stack of adhesive layers;
wherein the second adhesive layer comprises at least one seam to form a second pattern in the second adhesive layer; and
wherein:
the first pattern is different from the second pattern;
the first pattern is offset from the second pattern; and/or
the first pattern has a first size and the second pattern has a second size that is different from the first size.

18. The method of claim 17, wherein exposing the second adhesive layer is in response to separating the first portion of the first adhesive layer from the stack of adhesive layers.

19. The method of claim 17, wherein the first pattern is one of a spiral pattern, a double-back pattern, and a butterfly pattern.

20. The method of claim 17, wherein the first pattern is offset from the second pattern.
